(19) United States
(12) Patent Application Publication
(10) Pub. No.: US 2025/0049532 A1
(43) Pub. Date: Feb. 13, 2025

(54) SYSTEMS AND METHODS FOR MEDICAL IMAGING

(71) Applicant: Vita Group, LLC, Woodbury, MN (US)

(72) Inventors: Richard A. Thompson II, Saint Louis Park, MN (US); Jason Scherer, Woodbury, MN (US)

(21) Appl. No.: 18/797,625

(22) Filed: Aug. 8, 2024

Related U.S. Application Data
(60) Provisional application No. 63/531,952, filed on Aug. 10, 2023.

(51) Int. Cl. A61B 90/00; A61M 25/09

(52) U.S. Cl. CPC A61B 90/361; A61M 2025/09058; A61M 2025/09175

(57) ABSTRACT

Some systems, devices and methods detailed herein can include a guidewire equipped with one or more imaging sources and light sources positioned at a distal tip of the guidewire to provide medical imaging and illumination of a targeted body location.

### CROSS-REFERENCE TO RELATED APPLICATIONS

This application claims priority to U.S. Provisional Application No. 63/531,952, filed on Aug. 10, 2023. The disclosure of the prior application is considered part of, and incorporated by reference the disclosure of this application.

### TECHNICAL FIELD

This disclosure describes systems, devices, and methods for medical imaging, such as imaging and illumination of target locations via a guidewire instrument in body cavities, organs, and canals.

### BACKGROUND

Medical imaging can be utilized in a variety of medical procedures to provide visual inspection of areas in the body not otherwise visible to the medical professional. Some minimally invasive medical procedures utilize medical imaging to visually inspect a treatment area before, during, and after a treatment is administered to the treatment area. For example, some endoscopes or other imaging instruments can be inserted into treatment areas of a patient through a cannula or other working channel and provide a view of the treatment area to the medical personnel. Other imaging systems remain external to the patient and provide one or more views of treatment areas of the subject (e.g., via X-ray, ultrasound, magnetic resonance imaging).

### SUMMARY

This disclosure describes systems, devices, and methods for medical imaging, for example, imaging and illumination of target locations such as body cavities, organs, and canals. In particular implementations, the systems, devices, and methods described herein can include a guidewire (e.g., having an outer diameter of 2.5 mm or less, or optionally 2 mm or less) equipped with one or more imaging sources and light sources positioned at a distal tip of the guidewire to provide imaging and illumination of target locations. Optionally, the guidewire device can be implemented in a manner that is fully wireless (including wireless communication of image data to a display device in the surgical field), battery-powered, and sterile to provide rapid implementation and disposal during a medical procedure. For example, the systems, devices and methods described herein facilitate medical imaging during various medical procedures including gastroenterology, gynecology, urology, general surgery, car nose and throat (ENT), orthopedic surgery, plastic surgery, interventional radiology, among others. Optionally, the guidewire can be detachable from a handle body so that other medical instruments can be advanced over a proximal end of the guidewire (and then toward a distal end of the guidewire) while the detachable handle body is temporarily disconnected. In some examples detailed below, the handle body can include a power source, a wireless transmitter, and one or more electrical contacts that are configured to align with one or more mating contacts of the guidewire to power the one or more cameras and light sources and to transmit data communication between the one or more imaging sources (e.g., at the distal end of the guidewire) and the wireless transmitter (e.g., housed within the handle body). The connection between the wireless transmitter and the imaging source facilitates the convenient (and sanitary wire-free) transmission of image data acquired from the imaging source to a remote display, which can be in the form of a tablet device positioned in the surgical theater or display screen located outside of the sterile environment. Optionally, the system including the handle and the guidewire device equipped with one or more imaging sources and light sources can be a single-use, disposable instrument to be discarded after an individual use with a patient. Additionally, the transmission of the image data from the sterilized device can occur without wires or power cords extending to the device, thereby reducing the likelihood of contaminating the sterile environment.

Among other benefits, some systems and methods described herein can advantageously provide a more compact, sterilizable, and disposable approach to medical imaging of target areas of a patient. Additionally, some embodiments described in more detail below can achieve a compact and sterile solution that facilitates wireless transmission of image data from a sterilized imaging source to a remote display that is positioned outside of the sterile area. The sterilized imaging source is powered by a power source of a handle body that is also sterilized and not connected to components outside of the sterile environment.

Some embodiments described herein include a medical imaging system. The medical imaging system includes a handle including a handle body, a power source, a wireless transmitter, and a distal channel that extends through the handle body. The medical imaging system includes a guidewire configured to extend through the distal channel, the guidewire includes an imaging source and a light source positioned at a distal end of the guidewire, the imaging source and the light source are powered by the power source, the imaging source is in communication with the wireless transmitter, and the guidewire is detachable from the handle at the distal channel. The guidewire has a maximum outer diameter of 2 mm or less along an entire length of the guidewire extending distally of the handle, and the imaging source and the light source are housed within the maximum outer diameter of 2 mm or less at the distal end of the guidewire.

Such a system can include one or more of the following optional features. The medical imaging system where a proximal end of the guidewire includes a plurality of guidewire electrical contacts. The distal channel includes a plurality of handle electrical contacts positioned along an interior surface of the distal channel, the handle electrical contacts are configured to align with the guidewire electrical contacts. The guidewire electrical contacts include a plurality of conductive bands. The wireless transmitter wirelessly transmits image data acquired from the imaging source to a remote display. The system is disposable. The system is sterilizable. The system is a guidewire wireless system. The medical imaging system may include a lock connected to a distal end of the handle body around the distal channel, the lock releasably locks the guidewire to the handle. Responsive to unlocking the lock and detaching of the handle from the guidewire, one or more medical instruments are exchangeable over the guidewire. The system is a handheld cordless camera-equipped microguide.

Some embodiments described herein include a medical imaging method. The medical imaging method includes advancing a guidewire through a distal channel of a handle and to a target area of a patient, the guidewire includes an imaging source and a light source positioned at a distal end of the guidewire, the imaging source and the light source are housed within an outer diameter of the guidewire that is 2 mm or less along an entire length of the guidewire extending distally of the handle. The medical imaging method includes powering the imaging source and the light source by a power source at the handle. The medical imaging method includes capturing image data from the imaging source. The medical imaging method includes transmitting the image data from the imaging source to a wireless transmitter at the handle. The medical imaging method includes communicating the image data from the wireless transmitter to a remote display.

Such a method can include one or more of the following optional features. The medical imaging method where the guidewire diameter is constant along a length of the guidewire. The medical imaging method may include: detaching the guidewire from the handle; advancing a medical instrument over the guidewire and to the target area. The powering of the imaging source and the light source may include aligning a plurality of guidewire electrical contacts at a proximal end of the guidewire with a plurality of handle electrical contacts positioned along an interior surface of a distal channel of the handle. The method may include sterilizing the guidewire and the handle. The method may include disposing the guidewire and handle after a single use. The method may include activating the light source positioned at a distal end of the guidewire at a button on the handle. The method may include: locking the guidewire to the handle, unlocking the guidewire from the handle, and detaching the guidewire from the handle.

Some embodiments described herein include a medical imaging system. The medical imaging system includes a handle including: a handle body, a power source, a wireless transmitter, a distal channel that extends through the handle body. The medical imaging system includes a guidewire configured to extend through the distal channel and releasably attach to the distal channel at a proximal end of the guidewire, the guidewire has a guidewire diameter that is 2 mm or less along a length of the guidewire, the guidewire including: an imaging source positioned at a distal end of the guidewire and powered by the power source and in communication with the wireless transmitter, a light source positioned at a distal end of the guidewire and powered by the power source. The imaging source and the light source are positioned within the guidewire diameter; where the power source and the wireless transmitter are mounted to the handle body and the medical imaging system is sterilizable and disposable.

Particular implementations can, in certain instances, realize one or more of the following advantages. First, some embodiments described herein provide a compact solution that facilitates access to small target areas while providing robust and reliable image data from a distal end of a guidewire. Second, particular solutions described herein offer a readily sterilizable and disposable system that advantageously reduces contamination risk and potential non-sterile exposure within a sterile field. Third, some embodiments described herein provide wirelessly transmitted image data from a sterilized imaging source to a remote display that is positioned outside of the sterile area. Fourth, some embodiments described herein provide a multi-purpose device that facilitates imaging and illumination of target areas while also providing access to the target areas where the guidewire is detachable from the handle so that other medical instruments can be exchanged and advanced over the guidewire and to the target areas.

Fifth, some embodiments described herein advantageously provide a “guidewire wireless” device. As used herein “guidewire wireless” means that the sterile device both powers the imaging source at the distal end of the guidewire (e.g., via a battery source connected to the guidewire) without a power cable extending from the device and wirelessly transmits the image data acquired by the imaging source to a remote receiver without a data cable extending from the device. As such, the transmission of the image data from the sterilized device and the power supplied to the imaging/light source(s) are achieved without wires or power cords extending to components outside of the sterile environment.

Sixth, some embodiments described herein advantageously provide a “handheld cordless camera-equipped microguide” device. As used herein “handheld cordless camera-equipped microguide” means that the sterile device is steerable to navigate to a target area of a patient, powers the imaging source at the distal end of the microguide (e.g., via a battery source connected to the guidewire) without a power cable extending from the device, and wirelessly transmits the image data acquired by the imaging source to a remote receiver without a data cable extending from the device. As such, the handheld device facilitates steering of the microguide to the target area and transmission of the image data from the sterilized device and the power supplied to the imaging/light source(s) without wires or power cords extending to components outside of the sterile environment.

The details of one or more implementations are set forth in the accompanying drawings and the description below. Other features, objects, and advantages will be apparent from the description and drawings, and from the claims.

### DESCRIPTION OF DRAWINGS

FIG. 1 shows a perspective view of an example system for medical imaging, consistent with some embodiments of this disclosure.

FIG. 2 shows a side view of the medical imaging system of FIG. 1 along the line 2-2.

FIG. 3 shows a perspective view of a distal end of the medical imaging system of FIG. 1.

FIG. 4 shows a cross sectional view of the medical imaging system of FIG. 2 along the line 4-4.

FIG. 5 shows the cross-sectional view of FIG. 4, with a guidewire detached from a handle, consistent with some embodiments of this disclosure.

FIG. 6A shows the medical imaging system of FIG. 1 during operation of the system.

FIG. 6B shows the medical imaging system at another operational step.

FIG. 6C shows the medical imaging system at another operational step.

FIG. 6D shows the medical imaging system at another operational step.

FIG. 6E shows the medical imaging system at another operational step.

Like reference symbols in the various drawings indicate like elements.

### DETAILED DESCRIPTION

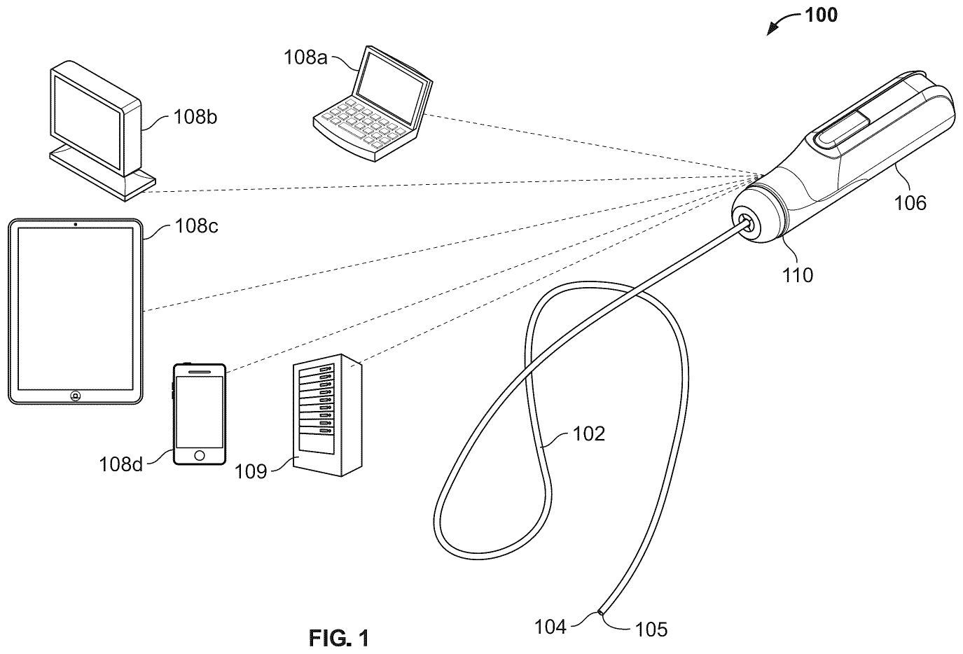

Referring to FIG. 1, some embodiments of a medical imaging system 100 can be implemented to access, illuminate, observe, and manipulate target areas of a patient during medical procedures. In this embodiment, the system 100 can include a guidewire 102, an image capture assembly 104 and a handle 106 that communicate with one or more remote displays 108a-d, and a server 109. In the depicted embodiment, the image capture assembly 104 is positioned at a distal end 105 of the guidewire 102 that is configured to be inserted into a target area of a patient. For example, the guidewire 102 is a flexible wire that is steerable (e.g., by via an actuator at the handle 106) to navigate to a target area of a patient. The guidewire 102 navigates to the target area and positions the image capture assembly 104 at or near the target area illuminate and capture image data (e.g., video, photo images, or other image data) of the target area. As described in more detail below, the image data acquired by the image capture assembly 104 is wirelessly transmitted from the handle 106 to one or more of the remote displays 108a-d, and optionally the server 109. In some optional implementations detailed below, the guidewire 102 can be detached from the handle 106 so that one or more medical instruments can be advanced over the guidewire 102 toward the target area. In some embodiments, the medical imaging system 100 can be utilized in at least gastroenterology, gynecology, urology, general surgery, car nose and throat (ENT), orthopedic surgery, plastic surgery, and interventional radiology procedures.

In this embodiment, the guidewire 102 can be connected to the handle 106 at a distal end 110 of the handle 106. In some embodiments, the guidewire 102 is flexible and a user (e.g., medical personnel) steers the guidewire 102 atraumatically within a patient's anatomy. The guidewire 102 can be steerable by an internal tension wire that controls the deflection of the guidewire 102 and the distal end 105 to facilitate navigation to and imaging of the target area. In some embodiments, the user could interact with a slider at the handle 106 (e.g., a slide on the top of the handle 106) that applies tension to a pull wire within the guidewire 102 to deflect the distal tip. The slider can be released to release the deflection of the guidewire 102. In some embodiments, the handle 106 includes a wheel that can be rotated (e.g., rotated proximally) to apply tension to a pull wire within the guidewire 102 to deflect the distal tip. The wheel can be rotated in an opposite direction (e.g., rotated distally) to release the deflection of the guidewire 102. In some embodiments, the handle 106 includes a button (e.g., button 140) that activates a motor (e.g., a motor housed in the handle body 120) to apply tension to a pull wire within the guidewire 102 to deflect the distal tip. The motor can be activated to rotate in an opposite direction to release the deflection of the guidewire 102. In some embodiments, the guidewire 102 and/or a distal section of the guidewire 102 is malleable, and the user shapes the guidewire 102 into a profile (e.g., a curved profile) could by bending the distal portion of the guidewire 102. The profile shaped by the user can create an angle that would spring a direction when inserted and advanced to a bifurcation or larger orifice. In some embodiments the guidewire 102 has a constant diameter along the length of the guidewire 102, including at the image capture assembly 104.

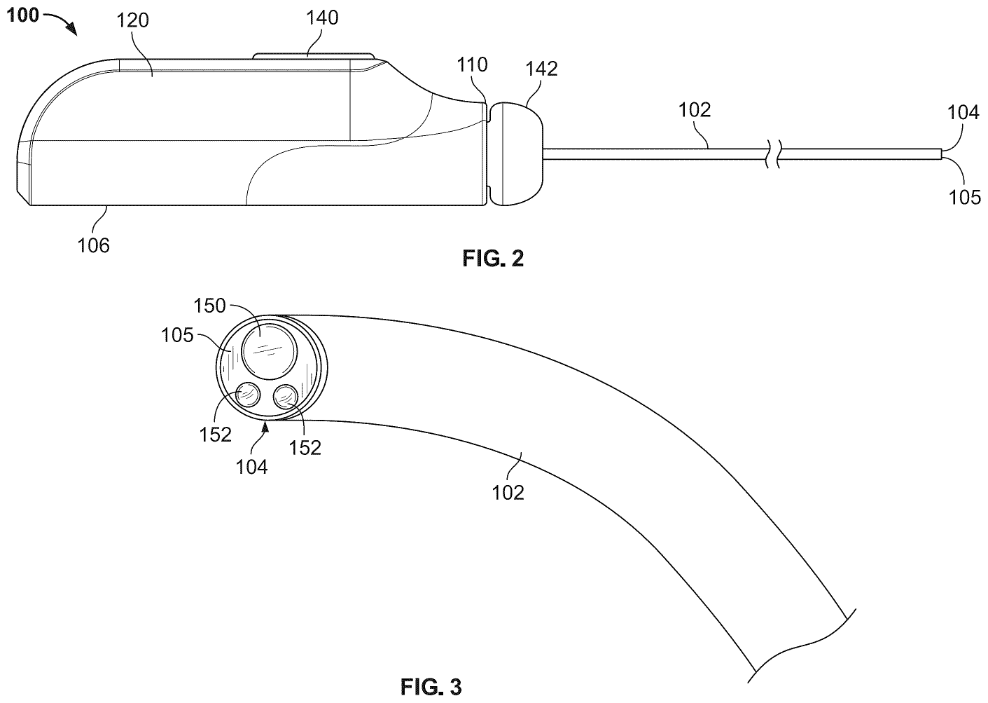

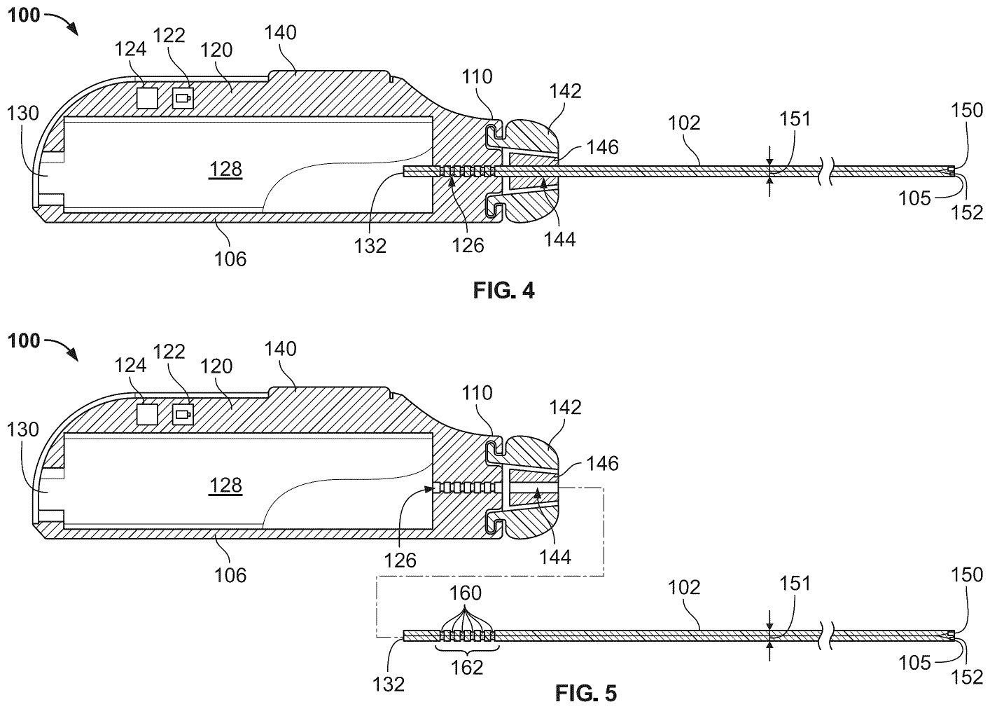

The image capture assembly 104 can be integrated within or attached to the distal end 105 of the guidewire 102. In either example, the image capture assembly has an equal or smaller diameter than the guidewire 102 to facilitate a compact solution capable of accessing anatomically small target areas. In some embodiments, the image capture assembly can include one or more imaging devices and one or more light sources. The one or more imaging devices and one or more light sources are discussed in further detail in reference to FIGS. 2-4.

The one or more remote displays 108a-d can include various screens that display the image data to the medical personnel. In some embodiments, the remote displays can include a laptop 108a, a monitor 108b, a personal display 108c, a smart phone 108d, or other displays. In some embodiments, the personal display 108 can be a smartphone, a tablet, a virtual reality headset, or other personal displays. In some embodiments, the smart phone 108d is a connected device that has a display and wireless communication capabilities. The one or more remote displays 108a-d can be positioned outside of a sterile environment, and display the image data from the guidewire wireless system in a sterile field. For example, the one or more remote displays 108a-d can be visible to medical personnel (E.g., in an operating room or outside an operating room), and outside of the sterile area. The image data can be a real time data stream illustrating the target area to one or more medical professionals during a procedure. The remote displays 108a-d can simultaneously display the image data, can individually display the image data, and can individually display separate segments of the image data. For example, the system 100 can capture real time image data that is streamed to one or more of the remote displays 108a-d. One display can illustrate past image data, while another display can illustrate current image data. In another example, two displays can show past image data (e.g., image data acquired during navigation to the target area), while another display illustrates the current and real-time image data. The server 109 can store image data acquired throughout the medical procedure.

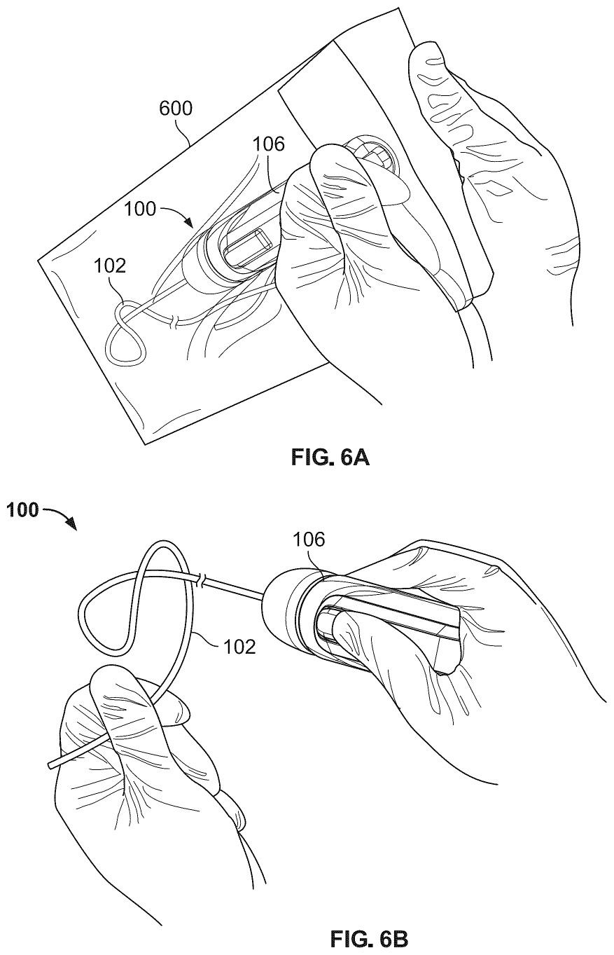

Referring to FIGS. 2-4, the handle 106 includes an ergonomic profile that facilitates control (e.g., one-handed control) of the handle 106 by medical personnel. In some embodiments, the handle 106 includes a handle body 120, a power source 122, a wireless transmitter 124, and a distal channel 126 that extends through the handle body 120. The handle body 120 defines an internal chamber 128 that is accessible via the distal channel 126 and a proximal opening 130. In some embodiments, the proximal opening 130 includes a larger opening than the inner diameter of the distal channel 126. In some embodiments, a proximal end 132 of the guidewire 102 extends into the internal chamber 128 as the guidewire 102 is advanced to the target area. For example, the guidewire 102 can be extended and retracted through the distal channel 126 and a length of the guidewire 102 extends proximally from the distal channel 126 and into the internal chamber 128. In some embodiments, the proximal end 132 of the guidewire 102 can extend through the proximal opening 130 of the handle body 120. For example, a length of the guidewire 102 including the proximal end 132 can extend through the internal chamber 128 and through the proximal opening 130 while and/or before the guidewire 102 navigates to a fully extended position (see e.g., FIG. 4). The handle 106 (and the components of the handle 106) can be sterilized using various sterilization practices. For example, the handle 106 can be sterilized by high-pressure steam (autoclave), dry heat (oven), chemical sterilants (glutaraldehydes, formaldehyde solutions), by physical agents (radiation), or other sterilization practices.

The handle 106 defines the distal channel 126 that extends from the distal end 110 of the handle 106 and into the internal chamber 128. In some embodiments, the guidewire 102 extends through and releasably connects to the distal channel 126. The distal channel 126 can include a channel diameter that is larger than the diameter of the guidewire 102 to facilitate extension and retraction of the guidewire 102 to and from the target area. The relative dimensions of the inner diameter of the distal channel 126 and the outer diameter of the guidewire 102 can facilitate a friction-fit connection between the guidewire 102 and the distal channel 126.

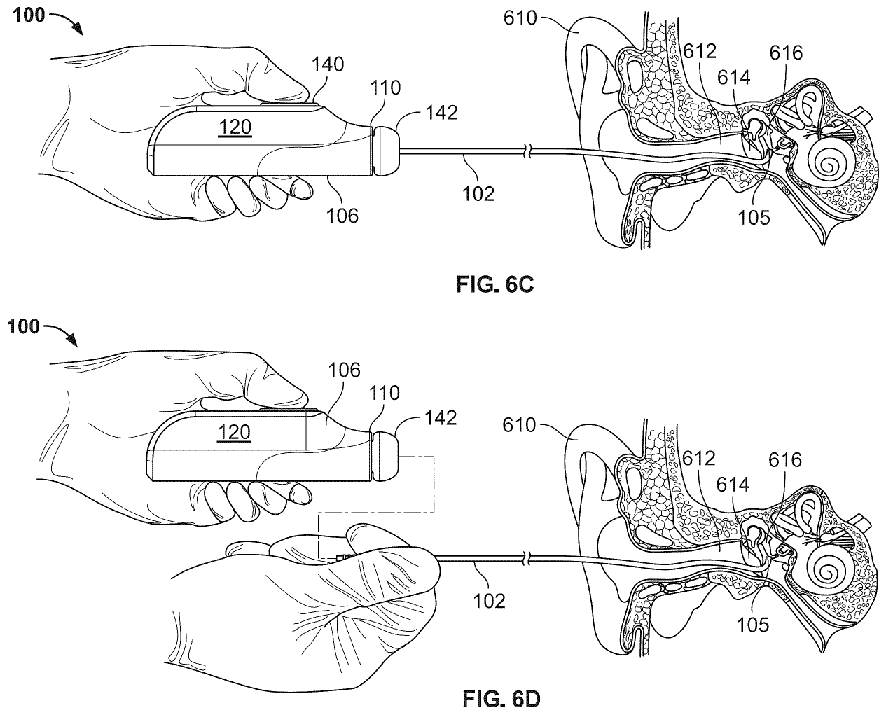

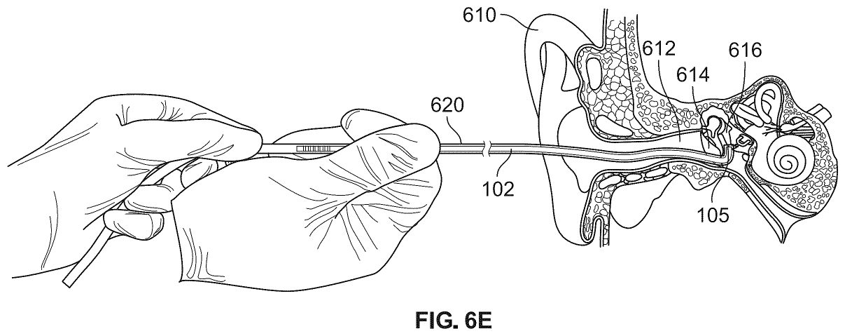

In some embodiments, the system 100 includes a lock 142 that connects the guidewire to the distal end of the handle. The lock 142 connects to the distal end 110 of the handle body 120 around the distal channel 126. The lock 142 defines a lock channel 144 that aligns with the distal channel 126, and the guidewire 102 can extend through the distal channel 126 and the lock channel 144. In some embodiments, the lock 142 is a twist lock that includes a collet 146 positioned around the lock channel 144 within the lock 142. The collet 146 is compressible responsive to the twisting of the lock 142 (e.g., via a threaded connection between the lock 142 and the distal end 110 of the handle 106) to compress against the guidewire 102 and lock the guidewire 102 in position within the distal channel 126 of the handle 106. The lock 142 is releasable by rotating the lock in an opposite direction to release the compression on the collet 146 and the guidewire 102 to facilitate detachment of the guidewire 102 from the handle 106 (see e.g., FIGS. 5 and 6A-E). In some embodiments, the lock 142 and the collet 146 are silicone to facilitate compressibility of the collet 146 responsive to the twisting of the lock 142.

Still referring to FIGS. 2-4, the handle 106 includes the power source 122. The power source 122 powers the system 100 (e.g., including the image capture assembly 104 including imaging source(s) and light source(s), the wireless transmitter 124, and electric navigation components). The power source 122 is mounted at the handle 106, and can be embedded within the handle body 120. In some embodiments, the power source 122 powers the system 100 without connection to power sources outside of the system 100. For example, power source 122 powers the system 100 without additional wires or power cords that could otherwise connect the system to components outside of the sterile environment. The power source 122 can be connected to the wireless transmitter 124, a button 140, and the distal channel via one or more electrical contacts (see e.g., FIG. 5). In some embodiments, the power source 122 is a battery that is disposable with the system 100. The power source 122 can be sterilized with the system 100. For example, the power source 122 can be installed at the handle 106 prior to the sterilization of the handle 106. The power source 122 can withstand and be sterilized by high-pressure steam (autoclave), dry heat (oven), chemical sterilants (glutaraldehydes, formaldehyde solutions), by physical agents (radiation), or other sterilization practices.

The handle 106 includes the wireless transmitter 124. The wireless transmitter 124 facilitates the wireless communication of the image data acquired by the image capture assembly 104 to the one or more remote displays 108a-d and/or the server 109. The wireless transmitter 124 is mounted at the handle 106, and can be embedded within the handle body 120. In some embodiments, the wireless transmitter 124 receives the image data from the image capture assembly 104 and transmits the image data to the one or more remote displays 108a-d and/or the server 109 without a physical connection to the remote displays 108a-d, the server 109, or other components outside of the sterile environment. The wireless transmitter 124 can be connected to the power source 122, the button 140, and the distal channel via one or more electrical contacts (sec e.g., FIG. 5). In some embodiments, the wireless transmitter 124 transmits the image data via short range wireless protocol (e.g., Bluetooth®), Wi-Fi, Near-Field Communication (NFC), radio-frequency transmission, among other wireless data transmission approaches. The wireless transmitter 124 facilitates the wireless uploading of image data from the image capture assembly 104 (e.g., such as still image and video files, video streaming, etc.). In some embodiments, the wireless transmitter 124 is disposable with the system 100. The wireless transmitter 124 can be sterilized with the system 100. For example, the wireless transmitter 124 can be installed at the handle 106 prior to the sterilization of the handle 106. The wireless transmitter 124 can withstand and be sterilized by high-pressure steam (autoclave), dry heat (oven), chemical sterilants (glutaraldehydes, formaldehyde solutions), by physical agents (radiation), or other sterilization practices.

In some embodiments, the handle 106 includes the button 140. The button 140 can be a push button that is user-selectable to control various operations of the system 100. For example, the user can actuate the button 140 to power the system 100 on or off (e.g., turning the battery from an off position to an on position that powers the system 100). The user can actuate the button 140 to sends instructions to the image capture assembly 104 to begin imaging. The user can actuate the button 140 to send instructions to the wireless transmitter 124 to send image data to the remote displays 108a-d and/or the server 109.

Still referring to FIGS. 2-4, the guidewire 102 is releasably attached to the handle 106 at the distal channel 126. The guidewire 102 extends through the distal channel 126 and extends distally from the handle 106 to navigate to the target area of the patient. The guidewire 102 can include a variety of lengths that facilitate extension and navigation through various anatomies to reach the target area. For example, the guidewire 102 can be between 300 mm and 2000 mm, between 400 mm and 1500 mm, between 500 mm and 1000 mm, between 600 mm and 900 mm. The guidewire 102 can include an outer diameter 151 that is 2 mm or less. In some embodiments the outer diameter is 2 mm or less, less than 2 mm, 1.7 mm or less, or 1.5 mm or less. The guidewire 102 has a constant outer diameter 151 along the length of the guidewire 102 (e.g., from the proximal end 132 to the distal end 105, including the image capture assembly 104.

The guidewire 102 includes the image capture assembly 104 housed within the distal end 105 of the guidewire. The image capture assembly 104 includes an imaging source 150 and a light source 152 that capture image data at the target area and illuminate the target area. In the illustrated embodiment, an imaging source 150 is shown with two light sources 152. In some embodiments, the image capture assembly 104 includes one, two, or three or more imaging sources 150. In some embodiments, the image capture assembly 104 includes one, two, or three or more light sources 152. The image capture assembly 104 is embedded within the outer diameter 151 of the guidewire 102. In some embodiments, the imaging source 150 has a smaller outer diameter than the outer diameter 151 of the guidewire 102. In some embodiments, the light source 152 has a smaller outer diameter than the outer diameter 151 of the guidewire 102 and the outer diameter of the imaging source 150.

In some embodiments, the imaging source 150 is a digital camera that captures still images and video from the target area. The still images and video from the target area are packaged as the image data that the wireless transmitter 124 communicates to the one or more remote displays 108a-d and/or server 109. In some embodiments, the imaging source 150 is a complementary metal oxide semiconductor (CMOS) image sensor, a charge coupled device (CCD), or other imaging source that has an outer dimension that can be housed within the outer diameter 151. The imaging source 150 is oriented to capture images and video distal of the distal end 105 of the guidewire at the target area. In some embodiments, the imaging source 150 captures images and video of the target area around the distal end 105 (e.g., directly distal of the distal end 105 and peripherally around the distal end 105) via a convex lens shape that facilities a wide-view of the target area.

The one or more light sources 152 illuminate the target area and facilitate improved image quality of the imaging source 150. The one or more light sources 152 are embedded within the outer diameter 151 of the guidewire 102 and are oriented to direct light outwardly from the distal end 105 (e.g., directly distal of the distal end 105 and peripherally around the distal end 105) to illuminate the target area. In some embodiments, the light sources 152 are light-emitting diodes (LEDs). For example, the image capture assembly 104 can include one light source 152, two light sources 152, three light sources 152, or more light sources 152 that are housed within the outer diameter 151 with the imaging source 150.

In some embodiments, the imaging source 150 and the light source 152 are powered by the power source 122. The power source 122 can be connected to the imaging source 150 and the light sources 152 via an electrical connection between the guidewire 102 and the handle 106 at the distal channel 126 (see e.g. FIG. 5). The electrical connection between the handle 106 and the guidewire 102 facilitates power transmission from the power source 122 to the imaging source 150 and the light sources 152. In some embodiments, the imaging source 150 is in communication with the wireless transmitter 124. For example, electrical connection between the handle 106 and the guidewire 102 facilitates communication of the image data from the imaging source 150 to the wireless transmitter 124. The connection between the imaging source 150 and the wireless transmitter 124 can be a wired connection (e.g., via electrical contacts at the distal channel 126 and the proximal end 132 of the guidewire shown in FIG. 5). In some embodiments, the wireless transmitter 124 transmits the image data from the imaging source 150 to the one or more remote displays 108a-d and/or the server 109 wirelessly.

In some embodiments, the guidewire 102 and the handle 106 are fully wireless, battery-powered (e.g., via power source 122), and sterile to provide rapid implementation and disposal during a medical procedure. For example, the guidewire 102 and the handle 106 facilitate medical imaging during various medical procedures including gastroenterology, gynecology, urology, general surgery, car nose and throat (ENT), orthopedic surgery, plastic surgery, interventional radiology, among others without physical connection (e.g., wired connection) to external devices such as power sources and/or displays. The guidewire 102 and handle 106 facilitate wireless communication of image data from the imaging source 150 to a display device (e.g., remote displays 108a-d) in the surgical field.

In some embodiments, the system 100 is a guidewire wireless system that is sterilizable and disposable. The imaging source 150 is sterilized and powered by the power source 122 the handle 106 that is also sterilized and not connected to components outside of the sterile environment. The image data acquired by the imaging source 150 is wirelessly transmitted from the system 100 and to the one or more remote displays 108a-d and/or server 109 that are positioned outside of the sterile area or not physically connected (e.g., via wires or otherwise) to the handle 106 or the guidewire 102. The transmission of the image data from the sterilized device and the power supplied to the imaging source(s) 150 and the light source(s) 152 occurs without additional wires or power cords that could otherwise connect the system 100 to components outside of the sterile environment.

Referring to FIG. 5, the guidewire 102 is detachable from the handle 106 in a manner that is configured to temporarily detach and then reconnect. For example, the guidewire 102 can be removed from the distal channel 126. The lock 142 is releasable by rotating the lock in an opposite direction to release the compression on the collet 146 and the guidewire 102 to facilitate detachment of the guidewire 102 from the handle 106. In some embodiments, the lock 142 and the collet 146 are rotated to release compression applied by the collet 146 on the guidewire 102 responsive to the twisting of the lock 142. With the lock 142 released, the guidewire 102 can be pulled out of the distal channel 126. In some embodiments, with the lock 142 released, the guidewire 102 can remain connected to the handle 106 at the distal channel 126. For example, friction between the distal channel 126 and the guidewire 102 can maintain the connection between the guidewire 102 and the distal channel 126. A user can apply a tensile force (e.g., in a proximal or distal direction) to pull the guidewire 102 out of the distal channel 126 with the lock 142 released.

The guidewire 102 includes a plurality of guidewire electrical contacts 160 along a proximal section 162 of the guidewire 102. The proximal section 162 of the guidewire 102 extends distally from the proximal end 132. In some embodiments, the proximal section 162 is spaced apart from the proximal end 132, and the proximal section 162 has a length that corresponds to a length of the distal channel 126. In some embodiments, the guidewire electrical contacts 160 are spaced apart from each other at regular intervals along the proximal section 162. The guidewire electrical contacts 160 can include a plurality of conductive bands that are housed within the outer diameter 151 of the guidewire 102 and facilitate an electrical connection between the imaging source 150, the light sources 152, and the proximal end 132 of the guidewire 102. In some embodiments, the electrical connection between the imaging source 150, the light sources 152, and the guidewire electrical contacts 160 can be a wired connection via a wire 161 that extends from the guidewire electrical contacts 160 to the imaging source 150 and the light sources 152.

The distal channel 126 includes a plurality of handle electrical contacts 170 positioned along an interior surface of the distal channel 126. The handle electrical contacts 170 are flush with the interior surface of the distal channel 126, and are configured to align with the guidewire electrical contacts 160 when the guidewire 102 is positioned in the distal channel 126. In some embodiments, the handle electrical contacts 170 are spaced apart from each other at regular intervals along distal channel 126. The handle electrical contacts 170 can include a plurality of conductive bands that facilitate an electrical connection between the power source 122, the wireless transmitter 124 and the distal channel 126. With the guidewire 102 positioned in the distal channel 126, the guidewire electrical contacts 160 and the handle electrical contacts 170 are aligned to facilitate an electrical connection and communication between the power source 122, the imaging source 150, and the light sources 152. With the guidewire 102 positioned in the distal channel 126, the guidewire electrical contacts 160 and the handle electrical contacts 170 are aligned to facilitate an electrical connection and communication between the wireless transmitter 124 and the imaging source 150.

Referring to FIGS. 6A-E, the system 100 can be implemented in an exemplary process for medical imaging. For example, FIG. 6A shows the system 100 after the system has been sterilized and the system 100 is fully assembled in a sterilized pouch 600. FIG. 6B shows the system 100 removed from the sterilized pouch 600, fully assembled with the guidewire 102 connected to the handle 106, sterile, and ready for use in the sterile field.

FIG. 6C shows the guidewire 102 inserted into an car 610 of a patient. For example, the guidewire 102 is steered through the car canal 612 and past the car drum. 614. The distal end 105 of the guidewire 102 (e.g., including the imaging source 150 and one or more light sources 152) is turned to orient the image capture assembly towards the target area (e.g., the auditory bones 616). Image data can be acquired during the navigation to the target area and once the target area is reached, and the image data can be wirelessly communicated to the one or more remote displays (e.g., remote displays 108a-d and/or the server 109). The target area can include target areas in various locations of the patient. The system 100 can be implemented in various medical procedures including gastroenterology, gynecology, urology, general surgery, car nose and throat (ENT), orthopedic surgery, plastic surgery, interventional radiology, among others.

Referring to FIG. 6D, the guidewire 102 is detached from the handle 106 while the distal end 105 of the guidewire 102 remains positioned at the target area. For example, the guidewire 102 can be removed from the distal channel 126 while remaining in position within the patient. In some embodiments, the user releases the lock 142 to facilitate detachment of the guidewire 102 from the handle 106.

Referring to FIG. 6E, a cannula 620 is exchanged over the guidewire 102 and to the target area with the handle 106 removed. In some embodiments, the guidewire 102 can serve as a guidewire that facilitates access to target areas within a patient, and one or more medical instruments can be exchanged over the guidewire. Some examples of medical instruments include cannulas, catheters, surgical tools, graspers, cutters, ablation tools, among other medical instruments.

While this specification contains many specific implementation details, these should not be construed as limitations on the scope of the disclosed technology or of what may be claimed, but rather as descriptions of features that may be specific to particular embodiments of particular disclosed technologies. Certain features that are described in this specification in the context of separate embodiments can also be implemented in combination in a single embodiment in part or in whole. Conversely, various features that are described in the context of a single embodiment can also be implemented in multiple embodiments separately or in any suitable subcombination. Moreover, although features may be described herein as acting in certain combinations and/or initially claimed as such, one or more features from a claimed combination can in some cases be excised from the combination, and the claimed combination may be directed to a subcombination or variation of a subcombination. Similarly, while operations may be described in a particular order, this should not be understood as requiring that such operations be performed in the particular order or in sequential order, or that all operations be performed, to achieve desirable results. Particular embodiments of the subject matter have been described. Other embodiments are within the scope of the following claims.

Accordingly, other implementations are within the scope of the following claims.

What is claimed is:

1. A medical imaging system, comprising:
a handle including a handle body, a power source, a wireless transmitter, and a distal channel that extends through the handle body; and
a guidewire configured to extend through the distal channel, the guidewire includes an imaging source and a light source positioned at a distal end of the guidewire, the imaging source and the light source are powered by the power source, the imaging source is in communication with the wireless transmitter, and the guidewire is detachable from the handle at the distal channel;
wherein the guidewire has a maximum outer diameter of 2 mm or less along an entire length of the guidewire extending distally of the handle, and the imaging source and the light source are housed within the maximum outer diameter of 2 mm or less at the distal end of the guidewire.

2. The medical imaging system of claim 1, wherein a proximal end of the guidewire includes a plurality of guidewire electrical contacts.

3. The medical imaging system of claim 2, wherein the distal channel includes a plurality of handle electrical contacts positioned along an interior surface of the distal channel, the handle electrical contacts are configured to align with the guidewire electrical contacts.

4. The medical imaging system of claim 2, wherein the guidewire electrical contacts include a plurality of conductive bands.

5. The medical imaging system of claim 1, wherein the wireless transmitter wirelessly transmits image data acquired from the imaging source to a remote display.

6. The medical imaging system of claim 1, wherein the system is disposable.

7. The medical imaging system of claim 1, wherein the system is sterilizable.

8. The medical imaging system of claim 1, wherein the system is a guidewire wireless system.

9. The medical imaging system of claim 1, further comprising a lock connected to a distal end of the handle body around the distal channel, the lock releasably locks the guidewire to the handle, wherein, responsive to unlocking the lock and detaching of the handle from the guidewire, one or more medical instruments are exchangeable over the guidewire.

10. The medical imaging system of claim 1, wherein the system is a handheld cordless camera-equipped microguide.

11. A medical imaging method comprising:
advancing a guidewire through a distal channel of a handle and to a target area of a patient, the guidewire includes an imaging source and a light source positioned at a distal end of the guidewire, the imaging source and the light source are housed within an outer diameter of the guidewire that is 2 mm or less along an entire length of the guidewire extending distally of the handle;
powering the imaging source and the light source by a power source at the handle;
capturing image data from the imaging source;
transmitting the image data from the imaging source to a wireless transmitter at the handle;
communicating the image data from the wireless transmitter to a remote display.

12. The medical imaging method of claim 11, wherein the guidewire diameter is constant along a length of the guidewire.

13. The medical imaging method of claim 11, further comprising:
detaching the guidewire from the handle;
advancing a medical instrument over the guidewire and to the target area.

14. The method of claim 11, wherein the powering of the imaging source and the light source comprises aligning a plurality of guidewire electrical contacts at a proximal end of the guidewire with a plurality of handle electrical contacts positioned along an interior surface of a distal channel of the handle.

15. The method of claim 11, further comprising sterilizing the guidewire and the handle.

16. The method of claim 15 further comprising disposing the guidewire and handle after a single use.

17. The method of claim 11 further comprising activating the light source positioned at a distal end of the guidewire at a button on the handle.

18. The method of claim 11 further comprising:
locking the guidewire to the handle;
unlocking the guidewire from the handle; and
detaching the guidewire from the handle.

19. A medical imaging system, comprising:
a handle including:
a handle body;
a power source;
a wireless transmitter;
a distal channel that extends through the handle body; and
a guidewire configured to extend through the distal channel and releasably attach to the distal channel at a proximal end of the guidewire, the guidewire has a guidewire diameter that is 2 mm or less along a length of the guidewire, the guidewire including:
an imaging source positioned at a distal end of the guidewire and powered by the power source and in communication with the wireless transmitter;
a light source positioned at a distal end of the guidewire and powered by the power source;
wherein the imaging source and the light source are positioned within the guidewire diameter;
wherein the power source and the wireless transmitter are mounted to the handle body and the medical imaging system is sterilizable and disposable.

20. The medical imaging system of claim 19, wherein the proximal end of the guidewire includes a plurality of guidewire electrical contacts and the distal channel includes a plurality of handle electrical contacts positioned along an interior surface of the distal channel, the handle electrical contacts are configured to align with the guidewire electrical contacts.
